(19) United States
(12) Patent Application Publication
(10) Pub. No.: US 2020/0308310 A1
(43) Pub. Date: Oct. 1, 2020

(54) NOVEL MODIFIED CELLULOSE, METHOD FOR PREPARING THE SAME AND USE THEREOF

(71) Applicant: Dongguan University of Technology, Dongguan, Guangdong (CN)

(72) Inventors: Dongliang RUAN, Dongguan, Guangdong (CN); Hui WANG, Dongguan, Guangdong (CN); Faliang CHENG, Dongguan, Guangdong (CN)

(21) Appl. No.: 16/608,793

(22) PCT Filed: Jul. 17, 2018
(86) PCT No.: PCT/CN2018/095903

§ 371 Date: Oct. 25, 2019

(30) Foreign Application Priority Data
Apr. 18, 2018 (CN) 201810349485.3
Apr. 18, 2018 (CN) 201810350234.7
Apr. 18, 2018 (CN) 201810350887.5

(51) Int. Cl. C08B 15/06; C08B 1/00; C07K 1/04

(52) U.S. Cl. CPC C08B 15/06; C07K 1/042; C08B 1/003

(57) ABSTRACT

A modified cellulose, method for preparing the same and use thereof. The structural formulas of the modified cellulose are shown in Formula (I), Formula (II) or Formula (III):

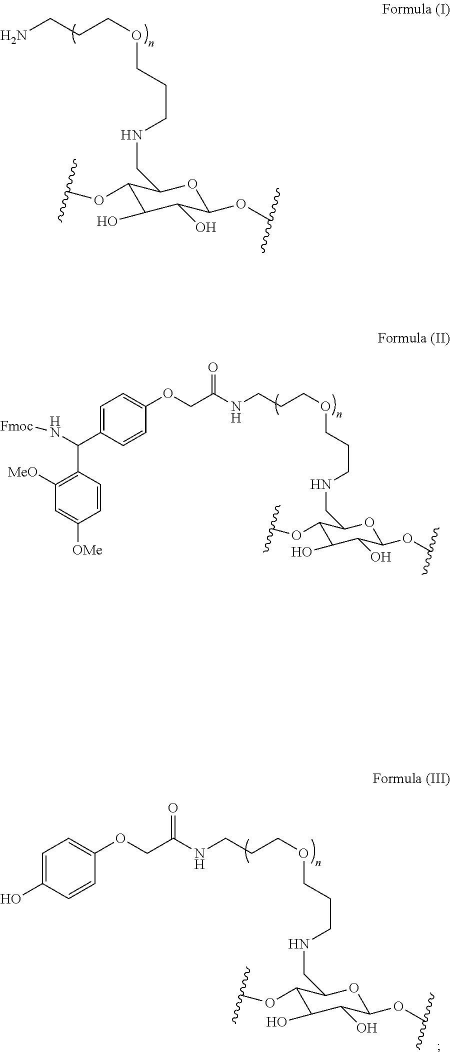

wherein, n is 2-7; the modified cellulose is obtained by substitution. The cellulose is substituted and modified with long-chain compounds containing amino terminal groups, so that the modified cellulose has space formed by long chains and amino terminal groups, which can be used as a matrix in solid-phase peptide synthesis (SPOT), to increase reaction activity and reaction space, reduce the difficulty of peptide synthesis, realize SPOT well, be suitable for synthesis in an automatic synthesizer, have high yield, and have low cost.

### TECHNICAL FIELD

The disclosure belongs to the field of matrix used in peptide synthesis, particularly to a novel modified cellulose, method for preparing the same and use thereof.

### BACKGROUND ART

Peptides are a class of bioactive compounds involved in different cellular functions in living things. They are a class of compounds whose molecular structures are between amino acids and proteins, made up of a variety of amino acids linked together by peptide bonds in a specific order. Chemical synthesis methods of peptides, such as liquid-phase and solid-phase methods, have been very mature. New peptides can be designed via total synthesis of peptides, which can be used to study the relationship between structure and function, provide important information about the reaction mechanism of peptide biosynthesis, establish enzyme models and synthesize new peptide drugs, etc. The total synthesis of peptides not only has great theoretical significance, but also important application value.

At present, solid-phase synthesis has become a common technique for peptide and protein synthesis, which has incomparable advantages over classical liquid-phase synthesis. For example, solid-phase peptide synthesis has the outstanding advantages such as saving time, labor and materials, easy computer control and easy popularization. The basic principle of solid-phase peptide synthesis is a process of repeated addition of amino acids, wherein the synthesis is from C-terminal (carboxyl-terminal) to N-terminal (amino terminal): firstly, the hydroxyl groups of amino acids containing hydroxyl-terminal groups in the synthesized peptide chains are covalently linked to insoluble polymer resins as solid-phase carriers; secondly, the amino acids bound to the solid-phase carriers are used as amino components to increase the peptide chains by removing amino protective groups and reacting with excessive activated carboxyl components, repeating (condensation→washing→deprotection→neutralization washing→the next round of condensation) operation to achieve the desired lengths of the synthesized peptide chains; finally, the peptide chains are cleaved from the resins, and after purification and other treatments, the desired peptides are obtained. Among them, the solid-state synthesis of alpha-amines protected by BOC (tert-butoxycarbonyl) is called BOC method, and the solid-state synthesis of alpha-amines protected by FMOC (9-fluorene methoxycarbonyl) is called FMOC method.

However, a large number of peptides are required to be synthesized, for example, the peptide chains are used for chemical screening and identification of multiligand protein affinity agents. Because the required peptides have light weights, and a large quantity of the peptides is needed, the technique of the above-mentioned standard solid-phase peptide synthesis on polymer resins is relatively slow and expensive. In recent years, solid-phase polypeptide synthesis based on traditional resins such as Wang resin and Rink resin has been developed several modified matrices to synthesize peptides. Among them, it includes tea-bag synthesis, digital photolithography, pin synthesis and SPOT synthesis on cellulose. These improved techniques are used to synthesize chemicals and build modules by using standard peptide synthesis in a very efficient way, and avoid the purification and analysis of peptides. Compared with standard solid-phase peptide synthesis, the cost of SPOT peptide synthesis is reduced dramatically. SPOT synthesis on cellulose is easy to be operated and can be performed manually, or semi-automatic or automatic robots can be used, the quantity and weights of peptides can be changed freely, and the operation for detection of peptide-ligand interaction is simple. The SPOT method follows the standard Fmoc chemistry and is based on solid-phase peptide synthesis on cellulose filters. Cellulose itself has several advantages over other materials: it is cheap and can tolerate organic solvents and acids used in peptide synthesis. In addition, cellulose is stable in aqueous solutions and non-toxic, so it is suitable for screening biological samples.

However, SPOT synthesis on cellulose also has some shortcomings and drawbacks, for example, cellulose is a polysaccharide composed of a linear-chain of hundreds to tens of thousands of β (1→4) linked D-glucose units, whose glycohydroxyl (—OH) groups and space formed by short chains lead to strict conditions for solid-phase peptide synthesis directly on the cellulose and low yield.

Therefore, the development of a modified cellulose as a matrix in solid-phase peptide synthesis to increase yields and make the synthesis easy, has important research significance and promotional value.

### SUMMARY OF THE INVENTION

The objective of the present disclosure is to overcome the shortcomings of the existing cellulose as a matrix in solid-phase peptide synthesis, such as harsh conditions and low yield, and to provide a novel modified cellulose. The modified cellulose, wherein the hydroxyl groups of glucose units in cellulose are substituted with long chains containing amino terminal groups, overcomes the problems caused by the hydroxyl groups of glucose units and space formed by short chains in cellulose, reduces the conditions for SPOT solid-phase peptide synthesis, increases yields and greatly reduces the cost of solid-phase peptide synthesis.

Another objective of the present disclosure is to provide a method for preparing the above-mentioned modified cellulose.

Another objective of the present disclosure is to provide the use of the above-mentioned modified cellulose as a matrix in solid-phase peptide synthesis.

In order to achieve the above purposes, the present disclosure adopts the following technical solutions:

A novel modified cellulose, the structural formulas of said modified cellulose are shown in Formula (I), Formula (II) or Formula (III):

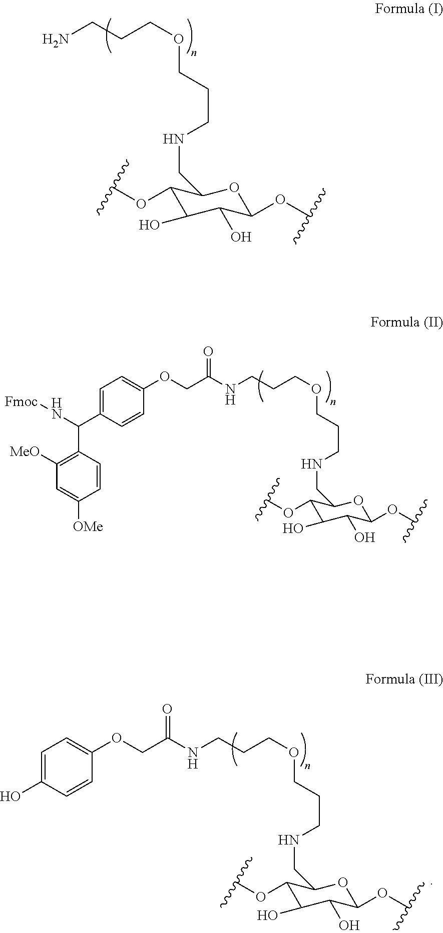

Wherein, n is 2-7; for example, n is 3;

the modified cellulose is obtained by substitution.

The present disclosure attempts to improve the properties of cellulose by enlarging the space formed by short chains with long-chain compounds, wherein the selection of functional groups and chain lengths of the long-chain compounds is a key factor. Many studies have found that when two terminal functional groups are amino groups, hydroxyl groups in the cellulose are substituted by one of the two amino terminal groups to form long-chain linkers; for the other terminal amino groups in the long chains, it provides the synthesis space to react with hydroxyl groups in amino acids, and at the same time it realizes peptide synthesis under the same reaction conditions, which makes the automated synthesis of peptides more efficient; furthermore, the simplicity and reproducibility of the repeating units in long-chain compounds also play an important role in improving their properties, wherein, if the chain lengths are too short (e.g. n=1), the lengths of peptides are limited; if the chain lengths are too long (e.g. n>7), peptide synthesis yields are low.

As an example, the structural formula of the modified cellulose is shown in Formula (II).

Based on the substitution with long-chain compounds, the modification is further done by using Rink Amide linker, by which the cellulose was modified to be used as a matrix in solid-phase peptide synthesis, and bioactive peptide screening can be performed without cleaving; at the same time, after the bioactive peptide screening, the synthesized peptides can be cleaved from the Rink Amide linker matrix for further determination of the synthesis by mass spectrometry, or quantitative analysis.

Therefore, the modified cellulose with Rink Amide linker provided in the disclosure, cellulose is substituted with long-chain compounds containing amino terminal groups, and then modified with Rink Amide linker, so that the cellulose has the space formed by long chains and higher reaction activity, and can be used as a matrix for better realizing both manual and automatic solid-state peptide synthesis with high yield and simple conditions.

As an example, the structural formula of the modified cellulose is shown in formula (III).

Based on substitution with long-chain compounds, the modification is further done by using Wang linker, by which the cellulose was modified to be used as matrix for solid-phase peptide synthesis, and bioactive peptide screening can be performed without cleaving; at the same time, after the bioactive peptide screening, the synthesized peptides can be cleaved from the Wang linker matrix for further determination of the synthesis by mass spectrometry, or quantitative analysis.

Therefore, the modified cellulose modified with Wang linker provided in the disclosure, cellulose is substituted with long-chain compounds containing amino terminal groups, and then modified with Wang linker, so that the cellulose has the space formed by long chains and higher solid-phase peptide synthesis activity, which can meet both manual and automatic instrumental synthesis. It can be used as a matrix for better realizing both manual and automatic solid-state peptide synthesis with high yield and simple conditions; at the same time, after peptide synthesis, bioactive peptide screening can be performed without cleaving, and after cleaving, the synthesized peptides can be provided qualitative and quantitative analysis.

According to the present disclosure, the cellulose can be paper-based cellulose.

For example, the cellulose is filter paper.

The present disclosure provides a method for preparing the above modified cellulose, comprising the following steps:

S1: mixing cellulose and a pyridine solution of p-toluenesulfonyl chloride, oscillating, washing and air-drying;

S2: adding long-chain compounds

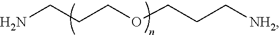

oscillating until the reaction is complete, washing, air-drying to obtain the modified cellulose of Formula (I);

or, the method further comprises the following steps of S3 and/or S4:

S3: adding Ring Amide resin and an activator to the modified cellulose of Formula (I), reacting and then washing to obtain the modified cellulose of Formula (II).

S4: adding Wang linker and an activator to the modified cellulose of Formula (I), reacting and then washing to obtain the modified cellulose of Formula (III).

According to the present disclosure, in step S1, the mass concentration of p-toluenesulfonyl chloride in the pyridine solution of p-toluenesulfonyl chloride is 350-400 g/L.

As an example, in step S1, the mass concentration of p-toluenesulfonyl chloride is 380 g/L.

According to the present disclosure, in step S3, the activator is N-hydroxybenzotriazole (HOBt) and N,N′-diisopropylcarbodiimide (DIC), or N-hydroxysuccinimide (HOSu) and N,N′-diisopropylcarbodiimide (DIC); the reaction temperature is 50-80° C., and the reaction time is 10-40 min.

According to the present disclosure, in step S3, the reaction temperature is 70° C., and the reaction time is 15 min.

According to the present disclosure, in step S4, the activator is hexamethylphosphoramide (HMPA) and 2-ethoxy-1-ethoxycarbonyl-1,2-dihydroquinoline (EEDQ); the reaction temperature is 18-25° C., and the reaction time is 12-16 h.

According to the present disclosure, in step S4, the reaction temperature is 20° C., and the reaction time is 14 h.

According to the present disclosure, before step S1 the method also comprises the step of waterless treatment of cellulose.

According to the present disclosure, in step S2, the solvents for washing are dimethylformamide and dichloromethane.

The present disclosure also provides the use of the above modified cellulose as a matrix in solid-phase peptide synthesis.

Compared with the prior art, the present disclosure has the following beneficial effects:

In the disclosure, cellulose is substituted with long-chain compounds containing amino terminal groups, so that the modified cellulose has the space formed by long chains and amino terminal groups, and can be used as a matrix in solid-phase peptide synthesis, thus increasing reaction activity and reaction space, reducing the difficulty of peptide synthesis, better realizing solid-phase peptide synthesis with high yield and low cost. In addition, when solid-phase peptide synthesis uses the modified cellulose further modified with Rink Amide linker as a matrix, bioactive peptide screening can be performed without cleaving; at the same time, after the bioactive peptide screening, the synthesized peptides can be cleaved from the Rink Amide linker matrix for further determination of the synthesis by mass spectrometry, or quantitative analysis; when the modified cellulose modified with Wang linker is used as a matrix in solid-phase peptide synthesis, enlarging the space formed by long chains and increasing reaction activity, it can meet both manual and automatic instrumental synthesis; it is better to realize solid-phase peptide synthesis with high yield and simple conditions; the bioactive peptide screening can be performed without cleaving, and after cleaving, the synthesized peptides can be provided qualitative and quantitative analysis after cleaving.

### BRIEF DESCRIPTION OF THE FIGURES

FIG. 1: Structure of a glucose unit in paper-based cellulose;

FIG. 2: Scheme of the modified paper-based cellulose of Example 1 and the preparation process;

FIG. 3: Scheme of the functional modification of paper-based cellulose and the peptide synthesis;

FIG. 4: (A) Sequences of synthesized peptides, (B) Intavis peptide synthesizer and UV absorption of the synthesized peptides with the modified paper-based cellulose as a matrix and (C) Western Blotting results;

FIG. 5: Scheme of the modified paper-based cellulose modified with Rink Amide linker of Example 4 and the preparation process;

FIG. 6: Determination of the synthesized peptides (Valine-Valine-Valine-Valine-Lysine) by liquid chromatography-mass spectrometry;

FIG. 7: Scheme of solid-phase peptide synthesis;

FIG. 8: Determination of the synthesized peptides (Valine-Valine-Valine-Valine-Lysine) by liquid chromatography-mass spectrometry.

### EXAMPLES

Hereinafter, the present invention is further described in detail with reference to the specific embodiments. The scope of the present disclosure is not limited in the following examples. It should be understood that a person skilled in the art can make various changes and modifications to the present disclosure without departing from the spirit and scope of the present disclosure.

In the following examples, the experimental methods without specifying conditions are usually in accordance with the conventional conditions in the art or the conditions recommended by the manufacturers; the raw materials, reagents, etc., used, unless otherwise specified, are all obtained from commercial approaches such as the conventional market.

### Example 1

The example provided a modified cellulose with the following structure:

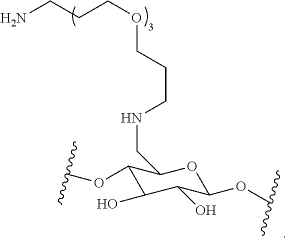

As shown in FIG. 2, the modified cellulose was prepared by the following method:

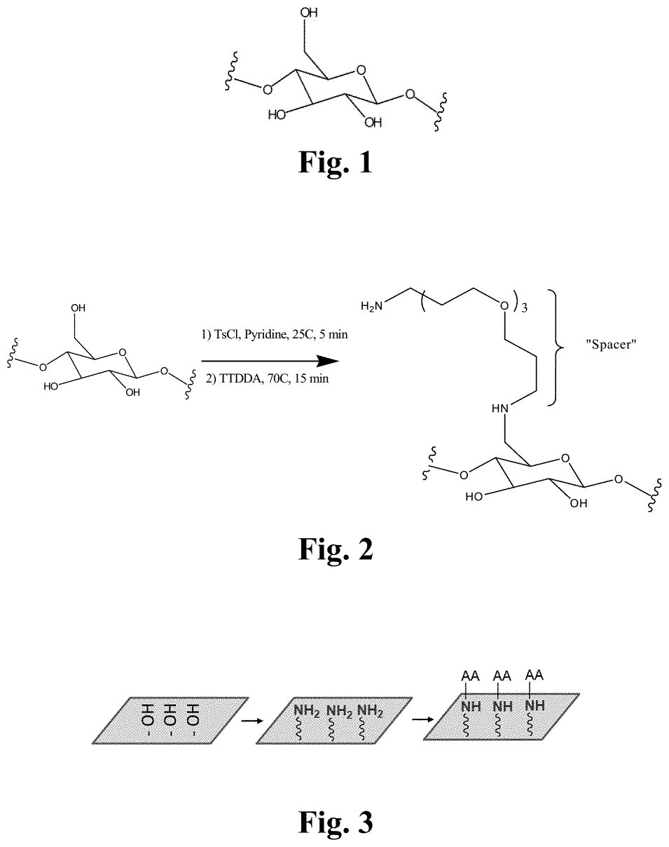

(1) prepare a certain size of lab filtration paper (Whatman 50, and the structure as shown in FIG. 1), place it in a stainless steel container, add enough dimethylformamide (DMF) (washing step) to the stainless steel container, oscillate gently for 1 hour, then pour out the solvent, repeat three times; and then add enough dichloromethane (DCM) to the stainless steel container, oscillate gently for 10 minutes, then pour out the solvent, repeat three times, and air-dry;

(2) place air-dried paper-based cellulose membrane in the stainless steel container, add enough pyridine solution of p-toluenesulfonyl chloride (380 g p-toluenesulfonyl chloride dissolved in 1 L pyridine) to the stainless steel container, oscillate gently at room temperature for 15 minutes, add enough DMF (washing step) to the stainless steel container, oscillate gently for 20 minutes, then pour out the solvent, repeat three times; add enough DCM to the stainless steel container, oscillate gently for 10 minutes, then pour out the solvent, repeat three times, and air-dry paper-based cellulose;

(3) place the air-dried paper-based cellulose membrane in the stainless steel container, add enough 4,7,10-trioxa-1,13-tridecanediamine (TTDDA), oscillate gently at room temperature overnight; add enough dimethylformamide (DMF) (washing step) to the stainless steel container, oscillate gently for 1 hour, then pour out the solvent, repeat three times; add enough dichloromethane (DCM) to the stainless steel container, oscillate gently for 10 minutes, then pour out the solvent, and repeat three times to obtain the modified cellulose.

### Example 2

The present example provided a modified cellulose with the following structure:

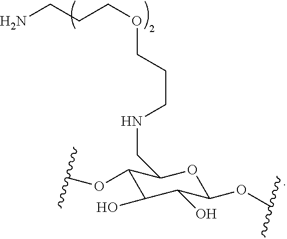

The modified cellulose was prepared by the following method:

(1) prepare a certain size of lab filtration paper (Whatman 50), place it in a stainless steel container, add enough dimethylformamide (DMF) (washing step) to the stainless steel container, oscillate gently for 1 hour, then pour out the solvent, repeat three times; and then add enough dichloromethane (DCM) to the stainless steel container, oscillate gently for 10 minutes, then pour out the solvent, repeat three times, and air-dry;

(2) place air-dried paper-based cellulose membrane in the stainless steel container, add enough pyridine solution of p-toluenesulfonyl chloride (350 g p-toluenesulfonyl chloride dissolved in 1 L pyridine) to the stainless steel container, oscillate gently at room temperature for 15 minutes, add enough DMF (washing step) to the stainless steel container, oscillate gently for 20 minutes, then pour out the solvent, repeat three times; add enough DCM to the stainless steel container, oscillate gently for 10 minutes, then pour out the solvent, repeat three times, air-dry paper-based cellulose;

(3) place the air-dried paper-based cellulose membrane in the stainless steel container, add enough bis(3-aminopropoxy)ethane, oscillate gently at room temperature overnight; add enough dimethylformamide (DMF) (washing step) to the stainless steel container, oscillate gently for 1 hour, then pour out the solvent, repeat three times; add enough dichloromethane (DCM) to the stainless steel container, oscillate gently for 10 minutes, then pour out the solvent, repeat three times to obtain the modified cellulose.

### Example 3

The present example provided a modified cellulose with the following structure:

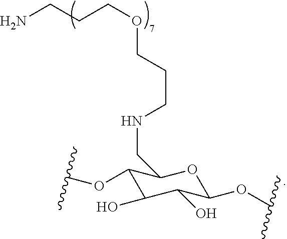

The modified cellulose was prepared by the following method:

(1) prepare a certain size of lab filtration paper (Whatman 50), place it in a stainless steel container, add enough dimethylformamide (DMF) (washing step) to the stainless steel container, oscillate gently for 1 hour, then pour out the solvent, repeat three times; and then add enough dichloromethane (DCM) to the stainless steel container, oscillate gently for 10 minutes, then pour out the solvent, repeat three times, and air-dry;

(2) place air-dried paper-based cellulose membrane in the stainless steel container, add enough pyridine solution of p-toluenesulfonyl chloride (400 g p-toluenesulfonyl chloride dissolved in 1 L pyridine) to the stainless steel container, oscillate gently at room temperature for 15 minutes, add enough DMF (washing step) to the stainless steel container, oscillate gently for 20 minutes, then pour out the solvent, repeat three times; add enough DCM to the stainless steel container, oscillate gently for 10 minutes, then pour out the solvent, repeat three times, air-dry paper-based cellulose;

(3) place the air-dried paper-based cellulose membrane in the stainless steel container, add enough hepta(ethylene glycol)bis(3-aminopropyl), oscillate gently at room temperature overnight; add enough dimethylformamide (DMF) (washing step) to the stainless steel container, oscillate gently for 1 hour, then pour out the solvent, repeat three times; add enough dichloromethane (DCM) to the stainless steel container, oscillate gently for 10 minutes, then pour out the solvent, repeat three times to obtain the modified cellulose.

### Example 4

The example provided a modified cellulose modified with Rink Amide linker, and the structure of the modified cellulose is as follows:

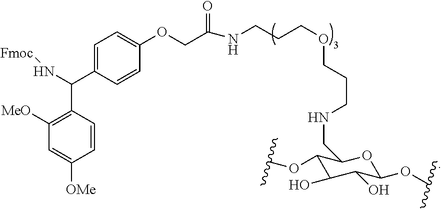

The modified cellulose provided in the example was prepared by the following method:

(1) the modified cellulose obtained according to the method in Example 1, air-dry;

(2) place the air-dried paper-based cellulose in step (1) in a stainless steel container, add 0.7 mol Rink Amide linker (structure:

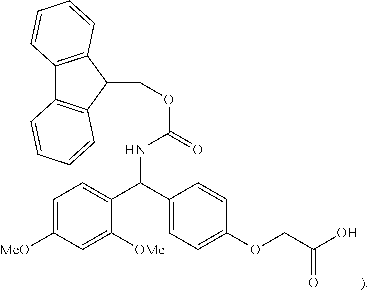 [SMILES: COc1ccc(C(NC(=O)OCC2c3ccccc3-c3ccccc32)c2ccc(OCC(=O)O)cc2)c(OC)c1]

0.7 mol hydroxybenzotriazole (HOBt), 0.7 mol N,N′-diisopropyl carboimide (DIC) and solvent DMF, oscillate at 70° C. for 15 minutes, then sequentially wash with DMF, ethanol (twice) and DCM, and air-dry to obtain the modified cellulose.

### Example 5

The example provided a modified cellulose modified with Rink Amide linker, and the structure of the modified cellulose is as follows:

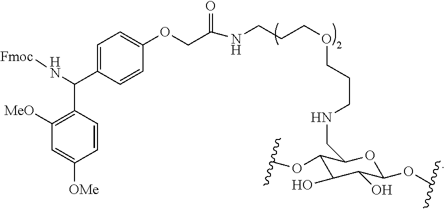

The modified cellulose was prepared by the following method:

(1) the modified cellulose obtained according to the method in Example 2, air-dry;

(2) place the air-dried paper-based cellulose in step (1) in a stainless steel container, add 0.7 mol Rink Amide linker (structure:

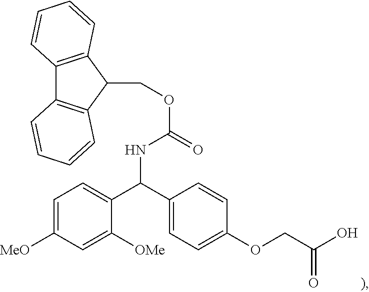 [SMILES: COc1ccc(C(NC(=O)OCC2c3ccccc3-c3ccccc32)c2ccc(OCC(=O)O)cc2)c(OC)c1]

0.7 mol hydroxybenzotriazole

(HOBt), 0.7 mol N,N′-diisopropyl carboimide (DIC) and solvent DMF, oscillate at 50° C. for 40 minutes, then sequentially wash with DMF, ethanol (twice) and DCM, and air-dry to obtain the modified cellulose.

### Example 6

The example provided a modified cellulose modified with Rink Amide linker, and the structure of the modified cellulose is as follows:

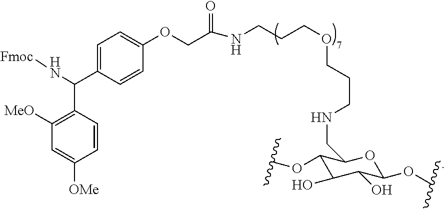

The modified cellulose was prepared by the following method:

(1) the modified cellulose obtained according to the method in Example 3, air-dry;

(2) place the air-dried paper-based cellulose in step (1) in a stainless steel container, add 0.7 mol Rink Amide linker (structure:

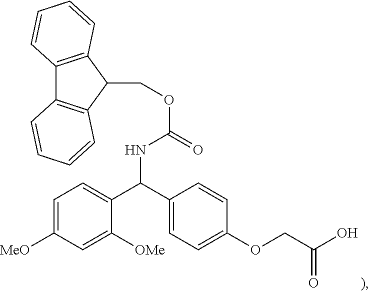 [SMILES: COc1ccc(C(NC(=O)OCC2c3ccccc3-c3ccccc32)c2ccc(OCC(=O)O)cc2)c(OC)c1]

0.7 mol hydroxybenzotriazole (HOBt), 0.7 mol N,N′-diisopropyl carboimide (DIC) and solvent DMF, oscillate at 80° C. for 10 minutes, then sequentially wash with DMF, ethanol (twice) and DCM, and air-dry to obtain the modified cellulose.

### Example 7

The example provided a modified cellulose modified with Wang linker, and the structure of the modified cellulose is as follows:

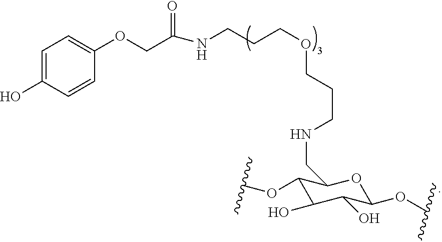

As shown in FIG. 2, the modified cellulose provided in the example was prepared by the following method:

(1) the modified cellulose obtained according to the method in Example 1, air-dry;

(2) place the air-dried paper-based cellulose in step (1) in a stainless steel container, add 0.1 mol Wang Linker, 0.11 mmol 2-ethoxy-1-ethoxycarbonyl-1,2-dihydroquinoline (EEDQ), 0.1 mol hexamethylphosphoramide (HMPA) and NMP (N-methylpyrrolidone) solvent, oscillate at 20° C. for 14 hours, then sequentially wash with DMF, ethanol (twice) and DCM, and air-dry to obtain the modified cellulose.

### Example 8

The example provided a modified cellulose modified with Wang linker, and the structure of the modified cellulose is as follows:

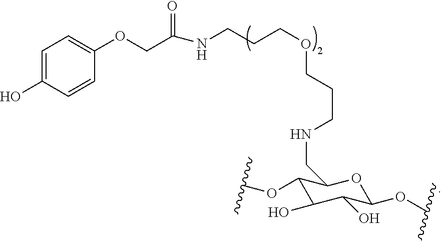

The modified cellulose was prepared by the following method:

(1) the modified cellulose obtained according to the method in Example 2, air-dry;

(2) place the air-dried paper-based cellulose in step (1) in a stainless steel container, add 0.1 mol Wang linker, 0.11 mmol 2-ethoxy-1-ethoxycarbonyl-1,2-dihydroquinoline (EEDQ), 0.1 mol hexamethylphosphoramide (HMPA) and NMP (N-methylpyrrolidone) solvent, oscillate at 20° C. for 14 hours, then sequentially wash with DMF, ethanol (twice) and DCM, and air-dry to obtain the modified cellulose.

### Example 9

The example provided a modified cellulose modified with Wang linker, and the structure of the modified cellulose is as follows:

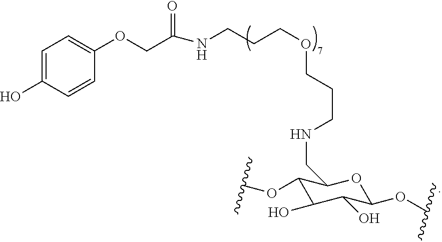

The modified cellulose was prepared by the following method:

(1) the modified cellulose obtained according to the method in Example 3, air-dry;

(2) place the air-dried paper-based cellulose in Step (1) in a stainless steel container, add 0.1 mol Wang Linker, 0.11 mmol 2-ethoxy-1-ethoxycarbonyl-1,2-dihydroquinoline (EEDQ), 0.1 mol hexamethylphosphoramide (HMPA) and NMP (N-methylpyrrolidone) solvent, oscillate at 20° C. for 14 hours, then sequentially wash with DMF, ethanol (twice) and DCM, and air-dry to obtain the modified cellulose.

### Application Example 1

Take the modified cellulose provided in Example 1 as an example, which was used as a matrix in solid-phase peptide synthesis, and test the properties of the synthesized products using UV absorption and Western Blotting.

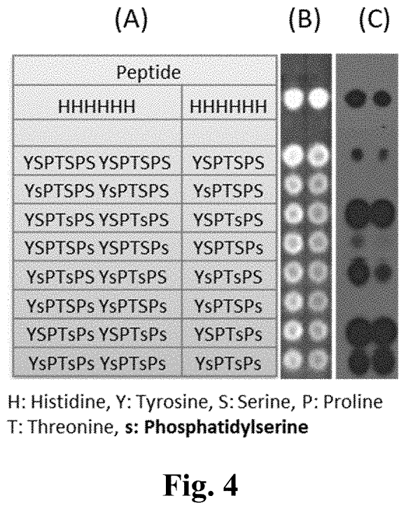

Peptide synthesizer was used to synthesize peptides in the direction from C-terminal to N-terminal. The schematic process of synthesis is shown in FIG. 3. The structures of the synthesized peptides are shown in FIG. 4 panel (A). Bioactive peptides containing six repeat histidine residues (HHHHHH) were used as a positive control for synthesis and detection. The synthesized peptides had the sequence tyrosine-serine-proline-threonine-serine-proline-serine (YSPTSPS), repeat structure (YSPTSPSYSPTSPS) and serine at positions 2,5,7 substituted with phosphatidylserine(s). FIG. 4 panel (B) is UV absorption of the synthesized peptides, all of which were positive, because the structures of the synthesized peptides were similar, they all absorbed light in the UV range, and there was no significant difference. FIG. 4 panel (C) is Western Blotting test results, because of the different amino acid sequences and biological activities of the peptides, in the Western Blotting tests, there were obvious differences due to the modification with amino acids (phosphatidylserine) at the different positions; even also in the peptides (YsPTsPS) with phosphatidylserine at positions 2 and 5, and their repeat structure (YsPTsPSYsPTsPS). It could be seen from the figures that the modified cellulose provided in the example could be used as a matrix in solid-state peptide synthesis, and the peptides had been successfully synthesized with the productivity and yields of the synthesis, which were suitable for bioactive peptide screening.

### Application Example 2

Take the modified cellulose modified with Rink Amide linker provided in Example 4 as an example, and test the properties.

Solid-phase peptide synthesis was carried out using the modified cellulose modified with Rink Amide linker as a matrix provided in Example 4, and the synthesized peptides were tested. The steps were as follows: synthesizing by using an automatic synthesizer on Rink Amide linker modified cellulose, then cleaving protective groups and the synthesized peptides from modified cellulose, finally analyzing the synthesized products by LC-mass spectrometry.

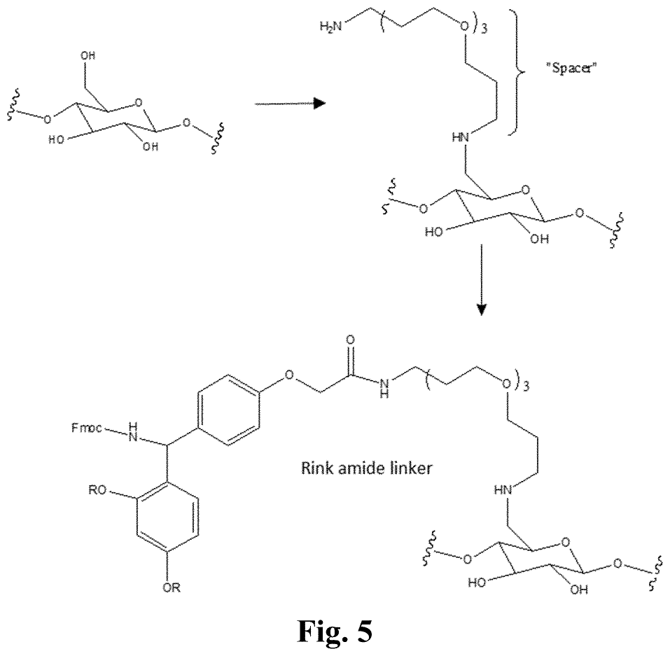

Peptide synthesizer was used to synthesize peptides in the direction from C-terminal to N-terminal. The schematic process of synthesis is shown in FIG. 5. Take the synthesized peptides Valine-Valine-Valine-Valine-Lysine (Ver-Ver-Ver-Ver-Lys) in an automatic synthesizer as an example: firstly, wetting and cleaning modified cellulose modified with Rink Amide linker with DMF and ethanol, removing Fmoc protection by using a pyridine solution, adding C-terminal amino acid Lys in the first step, and then repeating deprotection and synthesis steps until the desired peptides were synthesized; finally, deprotecting by using pyridine. Cleave synthesized peptides (such as Ver-Ver-Ver-Ver-Ver-Lys) from the modified cellulose modified with Rink Amide linker, cleave each SPOT peptides by using 100 μL of 90% trifluoroacetic acid, 3% triisopropylsilane, 2% water and 5% dichloromethane, and oscillate gently for 2 hours.

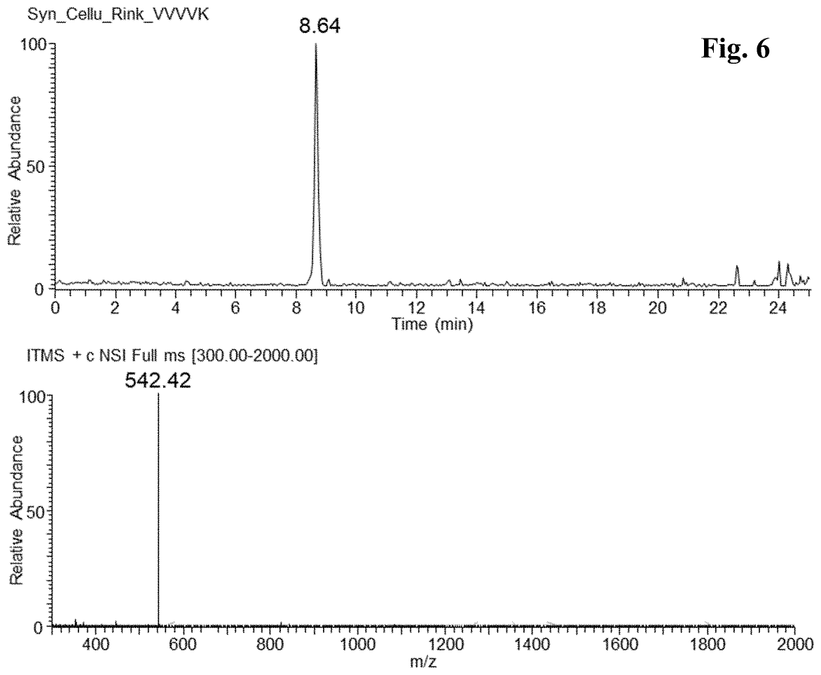

The synthesized peptides (Valine-Valine-Valine-Valine-Lysine) were determined by liquid chromatography-mass spectrometry (LC-MS). The molecular weight was 541.3. As shown in the LC-MS spectra (FIG. 6), the retention time of the main compound was 8.64 minutes, and the molecular ion peak was 542.4, which was the synthesized targeted peptide. As can be seen from FIG. 6, the peptides (Valine-Valine-Valine-Valine-Lysine) were successfully synthesized, and the modified cellulose had good properties, which could be used as a matrix in solid-phase peptide synthesis, exhibiting good reaction activity and high purity.

### Application Example 3

Take the modified cellulose modified with Wang linker provided in Example 7 as an example, and test the properties.

Solid-phase peptide synthesis was carried out using the modified cellulose modified with Wang linker as a matrix provided in Example 7, and the synthesized peptides were tested. The steps were as follows:

The steps were as follows: synthesizing by using an automatic synthesizer on Wang linker modified cellulos, then cleaving protective groups and the synthesized peptides from modified cellulos, then analyzing the synthesized products by LC-mass spectrometry.

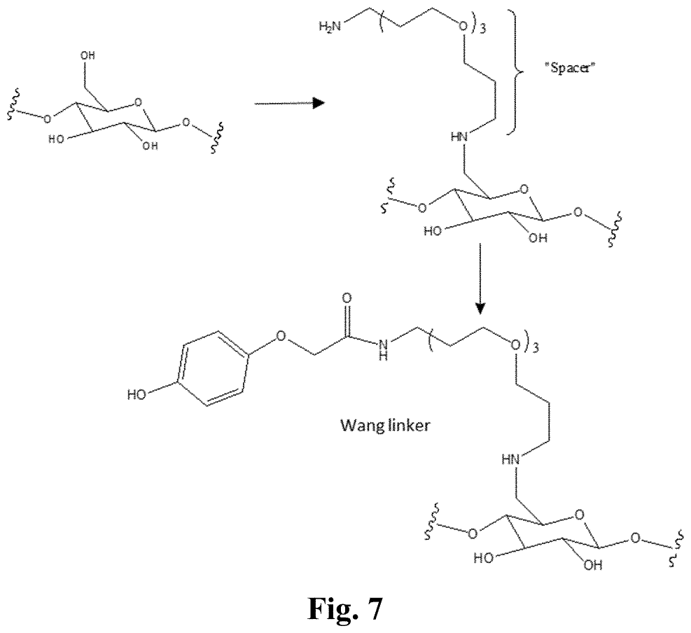

Peptide synthesizer was used to synthesize peptides in the direction from C-terminal to N-terminal. The schematic process of synthesis is shown in FIG. 7. Take the synthesized peptides Valine-Valine-Valine-Valine-Lysine (Ver-Ver-Ver-Ver-Lys) in an automatic synthesizer as an example: firstly, wetting and cleaning modified cellulose modified with Wang linker with DMF and ethanol, removing Fmoc protection by using a pyridine solution, adding C-terminal amino acid Lys in the first step, and then repeating deprotection and synthesis steps until the desired peptides were synthesized; finally, deprotecting by using pyridine. Cleave synthesized peptides (such as Ver-Ver-Ver-Ver-Ver-Lys) from the modified cellulose modified with Wang linker, cleave each SPOT peptide by using 100 μL of 90% trifluoroacetic acid, 3% triisopropylsilane, 2% water and 5% dichloromethane, and oscillate gently for 2 hours.

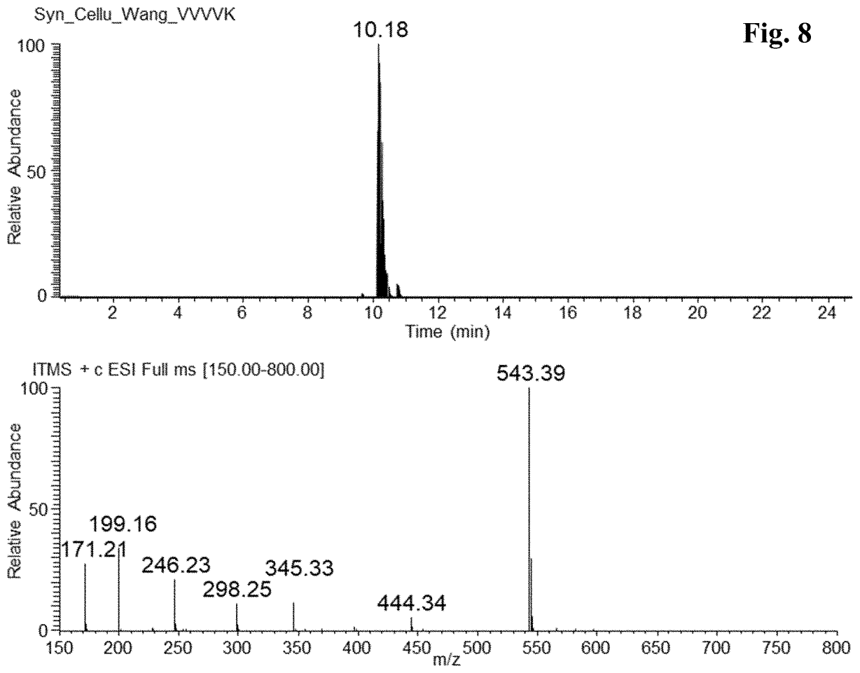

The synthesized peptides (Valine-Valine-Valine-Valine-Lysine) were determined by liquid chromatography-mass spectrometry (LC-MS). The molecular weight was 542.3. As shown in the LC-MS spectra (FIG. 8), the retention time of the main compound was 10.18 minutes, and the molecular ion peak was 543.39, which was the synthesized targeted peptide. As can be seen from FIG. 8, the peptides (Valine-Valine-Valine-Valine-Lysine) were successfully synthesized, and the modified cellulose had good properties, which could be used as a matrix in solid-phase peptide synthesis, exhibiting good reaction activity and high purity.

1. A novel modified cellulose, the structural formulas of said modified cellulose are shown in Formula (I), Formula (II) or Formula (III): 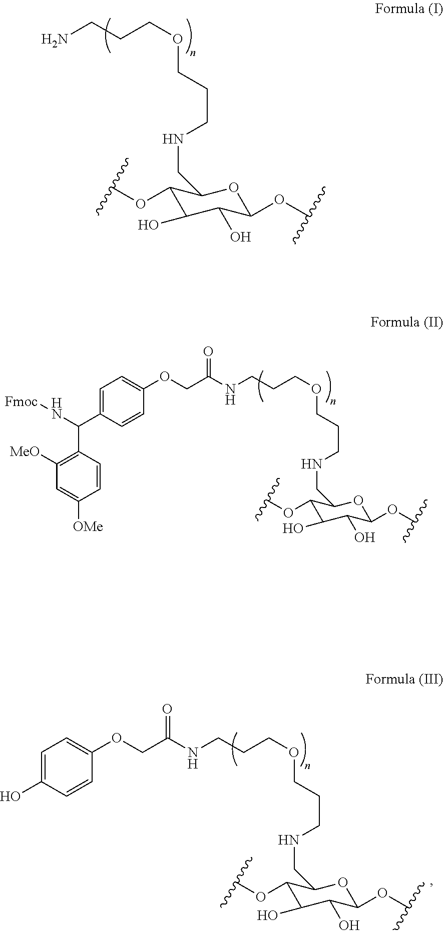
wherein, n is 2-7.

2. The modified cellulose according to claim 1, wherein, said cellulose is paper-based cellulose.

3. The modified cellulose according to claim 2, wherein, said paper-based cellulose is derived from cellulose filter papers.

4. The modified cellulose according to claim 1, wherein, n is 3.

5. A method for preparing the modified cellulose according claim 1, comprising the following steps:
S1: mixing cellulose and a pyridine solution of p-toluenesulfonyl chloride, oscillating, washing and air-drying;
S2: adding long-chain compounds 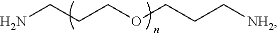
oscillating until the reaction is complete, washing, air-drying to obtain said modified cellulose of Formula (I).

6. The method for preparing the modified cellulose according to claim 5, further comprising the following steps of S3 and/or S4:
S3: adding Ring Amide resin and an activator to said modified cellulose of Formula (I), reacting and then washing to obtain said modified cellulose of Formula (II).
S4: adding Wang linker and an activator to said modified cellulose of Formula (I), reacting and then washing to obtain said modified cellulose of Formula (III).

7. The method for preparing the modified cellulose according to claim 5, in step S1, the mass concentration of p-toluenesulfonyl chloride in said pyridine solution of p-toluenesulfonyl chloride is 350-400 g/L.

8. The method for preparing the modified cellulose according to claim 7, said mass concentration of p-toluenesulfonyl chloride is 380 g/L.

9. The method for preparing the modified cellulose according to claim 6, in step S3, said activator is N-hydroxybenzotriazole (HOBt) and N,N′-diisopropylcarbodiimide (DIC), or N-hydroxysuccinimide (HOSu) and N,N′-diisopropylcarbodiimide (DIC); said reaction temperature is 50-80° C., and said reaction time is 10-40 min; in step S4, said activator is hexamethylphosphoramide (HMPA) and 2-ethoxy-1-ethoxycarbonyl-1,2-dihydroquinoline (EEDQ); said reaction temperature is 18-25° C., and said reaction time is 12-16 h.

10. The method for preparing the modified cellulose according to claim 5, before step S1 said method also comprises the step of waterless treatment of cellulose.

11. The use of the modified cellulose according to claim 1 as a matrix in solid-phase peptide synthesis.

12. A method for preparing the modified cellulose according to claim 2, comprising the following steps:
S1: mixing cellulose and a pyridine solution of p-toluenesulfonyl chloride, oscillating, washing and air-drying
S2: adding long-chain compounds 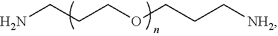
oscillating until the reaction is complete, washing, air-drying to obtain said modified cellulose of Formula (I).

13. A method for preparing the modified cellulose according to claim 3, comprising the following steps:
S1: mixing cellulose and a pyridine solution of p-toluenesulfonyl chloride, oscillating, washing and air-drying;
S2: adding long-chain compounds 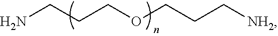
oscillating until the reaction is complete, washing, air-drying to obtain said modified cellulose of Formula (I).

14. A method for preparing the modified cellulose according to claim 4, comprising the following steps:
S1: mixing cellulose and a pyridine solution of p-toluenesulfonyl chloride, oscillating, washing and air-drying;
S2: adding long-chain compounds 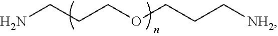
oscillating until the reaction is complete, washing, air-drying to obtain said modified cellulose of Formula (I).
